(19) United States
(12) Patent Application Publication
(10) Pub. No.: US 2011/0295087 A1
(43) Pub. Date: Dec. 1, 2011

(54) BIOLOGICAL INFORMATION DETECTION SENSOR, ELECTRIC APPARATUS USING THEREOF AND BIOLOGICAL INFORMATION DETECTION METHOD

(76) Inventors: Shigeki Shinoda, Tokyo (JP); Yasuhiro Sasaki, Tokyo (JP); Masatake Takahashi, Tokyo (JP); Hideki Ueda, Tokyo (JP)

(21) Appl. No.: 13/147,673

(22) PCT Filed: Feb. 4, 2010
(86) PCT No.: PCT/JP2010/000669

§ 371 Date: Aug. 3, 2011

(30) Foreign Application Priority Data
Feb. 4, 2009 (JP) 2009023908

(51) Int. Cl. A61B 5/00; G01N 33/48

(52) U.S. Cl. 600/306; 73/29.01

(57) ABSTRACT

A biological information detection sensor is provided by which it is possible to integrate multiple detection means, and it is possible to measure the biological information that is necessary in accordance with the usage state of the sensor used by the user while avoiding redundant information. The biological information detection sensor includes: a skin condition detection portion measuring at least one of a capacitance and an electric resistance change between engaged comb electrodes; a smell component detection portion measuring an electric resistance in accordance with objects absorbed on a selective sensitive membrane which is formed on a portion of the engaged comb electrodes; a temperature detection portion measuring an ambient temperature based on an electromotive force generated by a thermocouple which is formed by using at least two kinds of metallic materials and which is formed on a portion of the engaged comb electrodes; a switching means SW which compares the electromotive force generated by the thermocouple due to changes of the ambient temperature to a predetermined threshold, detects a contact state with an object having a heat source such as a biological body based on a comparison result and conducts switching operation of ON and OFF of the skin condition detection portion and the smell component detection portion based on the contact state.

### BACKGROUND OF THE INVENTION

1. Technical Field

The present invention relates to a biological information detection sensor, an electric apparatus using the sensor and a biological information detection method that are capable of measuring an amount of moisture, temperature and specific smelling components.

Priority is claimed on Japanese Patent Application No. 2009-023908, filed Feb. 4, 2009, the content of which is incorporated herein by reference.

2. Background Art

An amount of moisture and temperature of a skin constituted from both a surface skin and a demic layer are indices that indicate a skin condition of a person, and quantitative measure of a skin condition give important indices form a cosmetic and health control point of view. Further, particularly in recent years, a health control, mental condition, and measuring of smells, for example, alcohol and aging odors. It should be noted that a measuring method of amount of moisture is generally known in which, by using a detection element including engaged comb electrodes formed on a substrate, changes of the capacitance are measured that are caused in accordance with differences of conductivity between a sensor substrate and moisture. On the other hand, with respect to a measuring method of smells, a method is known in which changes of electric conductivity are measured that are caused by a physisorption on a sensitive membrane which has improved selectivity or by an oxidation reduction reaction of a sensitive membrane. Further, it is generally known that a mental condition of a person, for example the presence of stress or fatigue strongly affects perspiration and changes of temperature.
[Patent Document I] Japanese Patent Application, First Publication No. 2004-223263
[Patent Document 2] Japanese Patent Application, First Publication No. 2006-259156
[Patent Document 3] Japanese Patent Application, First Publication No. 2007-325842
[Patent Document 4] Japanese Patent Application, First Publication No. 2008-241318
[Patent Document 5] Japanese Patent Application, First Publication No. H07-012767
[Patent Document 6] Japanese Patent Application, First Publication No. H10-071130

### SUMMARY OF THE INVENTION

It is possible to detect cosmetics, changes of a body condition and changes of mental condition by measuring a skin condition of a person and by a long/short term surveillance of changes of the skin condition. When measuring a skin condition, physical quantities should be measured that have a strong relationship with the skin condition, and a comprehensive determination should be made based on multiple physical quantities, for example, the amount of moisture and temperature. Further, another method in which smelling components are measured is usable because it is possible to measure biological information without being injected or inserted. By measuring of smell components, it is possible to measure both aging odor which changes in accordance with age and degree of digestion of alcohol after drinking. However, when multiple physical quantities are measured, a detection element corresponding to each physical quantity is necessary, the number of components and the number of wirings of an overall sensor are increased, and a circuit area is enlarged. In addition, if independent detection elements are simply integrated, changes of multiple properties are simultaneously measured, and there is a problem in which it is not easy to measure biological information corresponding to a usage state of the sensor and a purpose of using the sensor.

The present invention is conceived to resolve such problems and has an object of providing a biological information detection sensor, an electric apparatus using the biological information detection sensor and a biological information detecting method that can integrate multiple detection portions and that can measure biological information that is required according to the usage state of the sensor used by a user.

A first solution for the above-described problems is a biological information detection sensor including: a smell detection means which senses a specific smell; a moisture detection means which senses moisture; a temperature detection means which detects an ambient temperature; and a switching means which switches between the smell detection means and the moisture detection means based on detection results of the temperature detection means.

Further, a second solution is a biological information detection sensor including: a skin condition detection portion measuring at least one of a capacitance and an electric resistance change between engaged comb electrodes; a smell component detection portion measuring an electric resistance in accordance with objects absorbed on a selective sensitive membrane which is formed on a portion of the engaged comb electrodes; a temperature detection portion measuring an ambient temperature based on a electromotive force generated by a thermocouple which is formed by using at least two kinds of metallic materials and which is formed on a portion of the engaged comb electrodes; and a switching portion which compares the electromotive force generated by the thermocouple due to changes of the ambient temperature to a predetermined threshold, detects a contact state with an object having a heat source such as a biological body based on a comparison result and conducts switching operation of ON and OFF of the skin condition detection portion and the smell component detection portion based on the contact state.

Further, a third solution is an electric apparatus including one of the biological information detection sensors of the above-described solutions, wherein the electric apparatus conducts a measuring operation of a skin condition and a breath odor of a person based on detection results of the biological information detection sensor.

Further, a third solution is an electric apparatus including one of the biological information detection sensors of the above-described solutions, wherein the biological information detection sensor is mounted at immersible points such as a gap between chassis, and the electric apparatus detects immersion into the electric apparatus based on detection results of the biological information detection sensor.

Further, a fourth solution is an electric apparatus including one of the biological information detection sensors of the above-described solutions, wherein the electric apparatus simultaneously or gradually conducts both detection of abnormal heat from circuit components mounted on a circuit board and/or a rechargeable battery and detection of smoke which is an indication before a fire based on detection results from the biological information detection sensor.

Further, a fifth solution is an electric apparatus including one of the biological information detection sensors of the above-described solutions, wherein the electric apparatus detects an approaching human body based on temperature detection results of the biological information detection sensor and accurately detects the human body by measuring an electric impedance of the human body based on moisture detection results.

Further, a sixth solution is a biological information detection method including: a detection operation of detecting a specific smell; a moisture detection operation of detecting moisture; a temperature detection operation of detecting an ambient temperature; and a switching operation of switching the smell detection operation and the moisture detection operation based on detection results of the temperature detection operation.

By using the above-described solutions, an advantage is obtained in which it is possible to integrate multiple detection means, and in addition, it is possible to measure the biological information that is necessary in accordance with the usage state of the sensor used by the user while avoiding redundant information. Further, in accordance with the above-described solutions, it is possible to reduce a number of components, a circuit area and a number of wirings, and in addition, it is possible to produce a biological information detection sensor which consumes comparatively little electric power.

### BRIEF DESCRIPTION OF THE DRAWINGS

FIG. 1A is a plane figure showing a constitution of a biological information detection sensor of one embodiment.

FIG. 1B is a cross-sectional figure along with a line between A-A′ showing a constitution of a biological information detection sensor of one embodiment.

FIG. 2 is an equivalent electric circuit diagram of the biological information detection sensor.

FIG. 3 is other electric circuit diagram of the biological information detection sensor.

FIG. 4 is a drawing for explaining operations of the biological information detection sensor.

FIG. 5A is a drawing for explaining an embodiment of the biological information detection sensor.

FIG. 5B is a drawing for explaining an embodiment of the biological information detection sensor.

FIG. 6A is a perspective drawing showing a constitution of an electric apparatus (mobile terminal) to which the biological information detection sensor is applied.

FIG. 6B is a plane figure showing a constitution of an electric apparatus (mobile terminal) to which the biological information detection sensor is applied.

FIG. 6C is a cross-sectional figure showing a constitution of an electric apparatus (mobile terminal) to which the biological information detection sensor is applied.

FIG. 7 is a drawing for explaining operations of the electric apparatus.

FIG. 8 is a drawing for explaining operations of the electric apparatus.

FIG. 9A is a perspective drawing showing a constitution of other electric apparatus (mobile terminal) to which the biological information detection sensor is applied.

FIG. 9B is a plane figure showing a constitution of other electric apparatus (mobile terminal) to which the biological information detection sensor is applied.

FIG. 9C is a cross-sectional figure showing a constitution of other electric apparatus (mobile terminal) to which the biological information detection sensor is applied.

FIG. 10 is a drawing for explaining operations of the electric apparatus.

FIG. 11 is a drawing for explaining operations of the electric apparatus.

FIG. 12A is a perspective drawing showing a constitution of an electric apparatus (mobile terminal) other than above to which the biological information detection sensor is applied.

FIG. 12B is a plane figure showing a constitution of an electric apparatus (mobile terminal) other than above to which the biological information detection sensor is applied.

FIG. 12C is a cross-sectional figure showing a constitution of an electric apparatus (mobile terminal) other than above to which the biological information detection sensor is applied.

### DETAILED DESCRIPTION OF THE INVENTION

### Embodiment 1

Hereinafter, in reference to the drawings, one embodiment is explained (First embodiment).

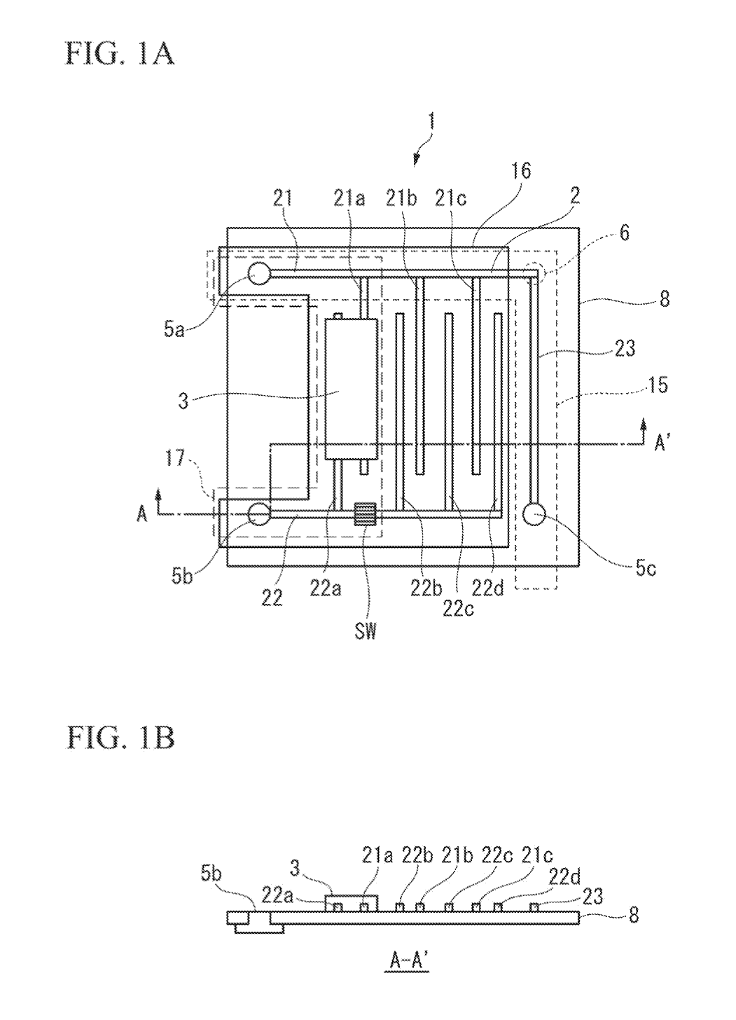

FIG. 1A is a plane figure showing a constitution of a biological information detection sensor (hereinafter, sensor) 1 of one embodiment. FIG. 1B is a cross-sectional figure along with a line between A-A′ of the biological information detection sensor 1. In these drawings, a substrate 8 is in a square shape and made from a glass-ceramic, and a engaged comb electrode 2 is formed on the substrate 8. The engaged comb electrode 2 includes: a first comb electrode constituted from both a horizontal electrode 21 in a narrow platy shape and vertical electrodes 21a-21c in a narrow platy shape which are arranged at even intervals; and a second comb electrode constituted from both a horizontal electrode 22 in a narrow platy shape and vertical electrodes 22a-22c in a narrow platy shape which are arranged at even intervals, and the vertical electrodes 21a-21c and the vertical electrodes 22a-22c are alternatively arranged at even intervals. Further, a vertical electrode 23 is formed at a lateral portion of the vertical electrode 22d. A skin condition detection portion 16 is constituted from the vertical electrodes 21a-21c and 22a-22d and horizontal electrodes 21 and 22.

Further, an electrode terminal 5a is formed on an end of the horizontal electrode 21, the other end of the horizontal electrode 21 is connected to an end of the vertical electrode 23, and an electrode terminal 5c is formed on the other end of the vertical electrode 23. An electrode terminal 5b is formed on an end of the horizontal electrode 22. The above-described engaged comb electrode 21 is made from copper, and the vertical electrode 23 is made from a copper nickel alloy. In accordance with such a constitution, a connection portion 6 between the horizontal electrode 21 and the vertical electrode 23 forms a thermocouple, and the temperature detection portion 15 is constituted from the horizontal electrode 21 and the vertical electrode 23.

A sensitive membrane 3 is attached to an upper side of the substrate 8 in a manner in which the sensitive membrane 3 covers the vertical electrodes 21a and 22a. The sensitive membrane 3 changes its electric resistance when absorbing particular smell components, and a smell component detection portion 17 is constituted from the sensitive membrane 3, the vertical electrodes 21a and 22a and horizontal electrodes 21 and 22. The horizontal electrodes 22 are divided at a center portion, in particular, at a position where the vertical electrodes 22a and 22b are attached, and a switch means SW is set at the divided portion. The switch means SW is controlled to be turned on/off based on detection results of the temperature detection portion 15.

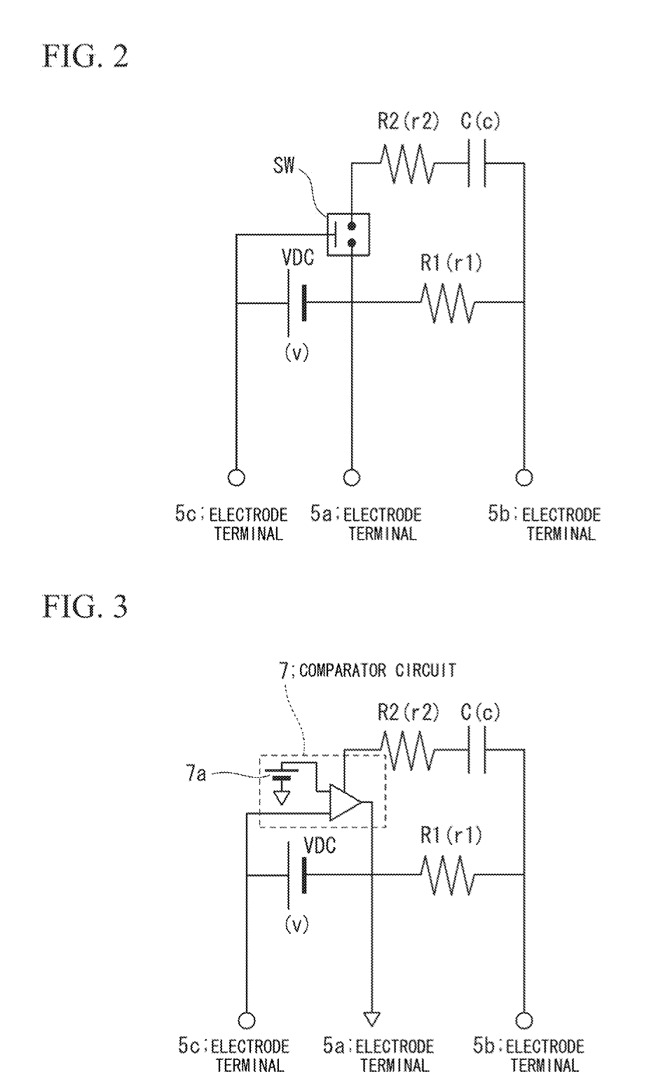

FIG. 2 shows an equivalent electric circuit of the above-described sensor 1. In this drawing, a resistance R1 indicates the smell component detection portion 17 shown in FIG. 1A, and a directly connected circuit including both a resistance R2 and a capacitor C indicates the skin condition detection portion 16. A direct current voltage source VDC indicates an electromotive force of the thermocouple (temperature detection portion 15) and is changed due to the ambient temperature. The resistance R1 is an electric resistance between the vertical electrodes 21a and 22a that are covered by the sensitive membrane 3 and that a resistance change r1 of the sensitive membrane is caused when adhering and/or removing the smell components. Further, a capacitor C has a capacitance between the first and second comb electrodes and causes a capacitance fluctuation c due to water adhesion. The direct current voltage source VDC generates an electromotive force v in accordance with changes of an ambient temperature. Further, the electrode terminal 5c which constitutes the thermocouple is connected to the switch means SW. It should be noted that it is possible to form the switch means SW as a comparator circuit 7 on a substrate 8 as shown in FIG. 3 for conducting a comparing operation based on a reference voltage 7a, or it is possible that the switch means SW conduct a switching operation between the skin condition detection portion 16 and the component detection portion 17 by conducting a comparing operation based on a reference voltage by using software.

Hereinafter, an operation of the sensor 1 of the above-described embodiment is explained.

The sensor 1 automatically determines a condition of contact to a measuring object based on changes of temperature and can measure temperature, a skin condition and a particular smell component by using a single element. The sensor 1 measures changes of the temperature around the thermocouple based on an electromotive force (v) of the thermocouple and measures the particular smell component based on the resistance change r1 of the sensitive membrane 3. In addition, the skin condition is measured based on both the capacitance fluctuation c between the comb electrodes and changes r2 of the resistance R2. Here, one of the skin condition detection portion 16 and the smell component detection portion 17 is selected based on the condition of contact to the measuring object by using the switch means (SW) which conducts ON/OFF operation in accordance with the electromotive force v. The reference voltage which is a threshold can be arbitrarily set. Further, an alternating voltage is applied between the electrode terminals 5a-5b, and both the resistance change r1 and the capacitance fluctuation c is measured as fluctuation of the current.

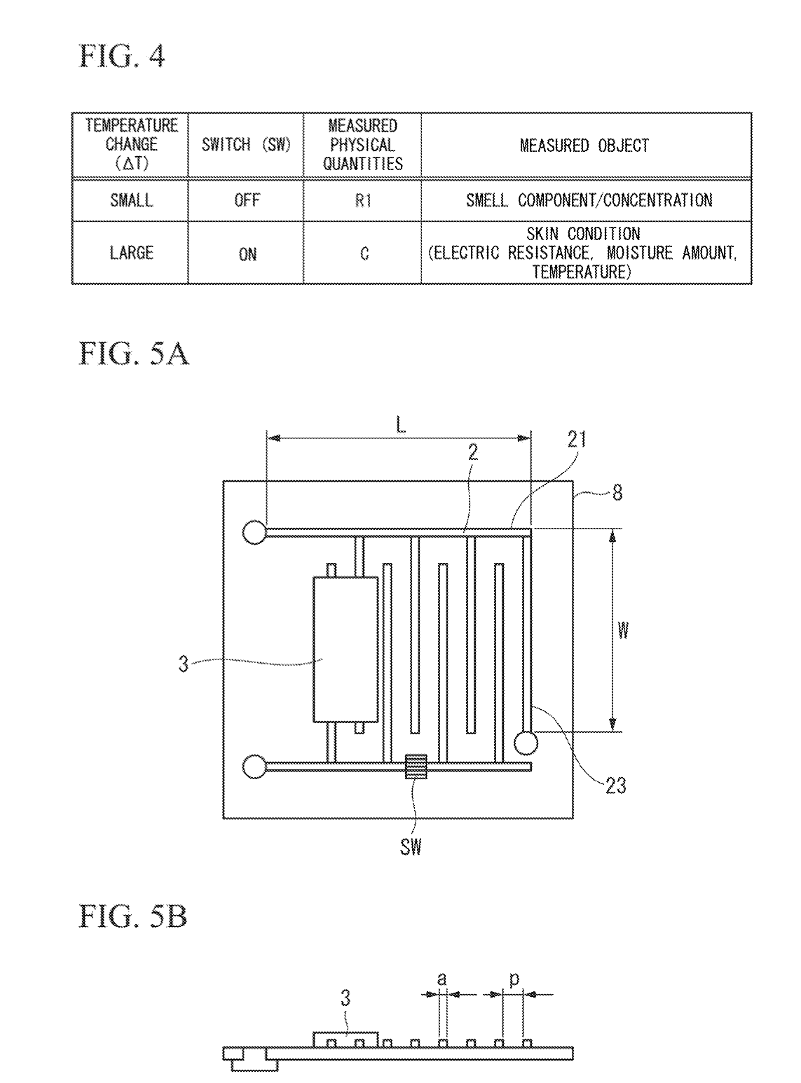

As shown in FIG. 4, when the electromotive force v is smaller than a predetermined threshold, in other words, when a temperature change (ΔT) is minimal, if the switch means (switch) SW is in an OFF state, a current is generated between the terminal electrodes 5a-5b in accordance with the applied voltage and the resistance change r1 of the smell component detection portion 17. On the other hand, when the electromotive force v is larger than the predetermined threshold, in other words, when a temperature change is large, if the switch means (switch) SW is in a ON state, a current is generated between the terminal electrodes 5a-5b in accordance with the applied voltage and the capacitance fluctuation c of the skin condition detection portion 16. It is acceptable to set the switch means (switch) ON when the temperature change is large, to set the switch means OFF when the temperature change is minimal, to measure the resistance R1 which is the smell component detection portion 17 when the temperature change is large, and to measure the capacitor C which is the skin condition detection portion 16 when the temperature change is minimal.

As described above, in accordance with the above-described sensor 1, by using the thermocouple using a portion of the engaged comb electrode 2 as a switch means between the skin condition detection portion 16 and the smell component detection portion 17, it is possible to automatically detect the touching condition to the object based on changes of ambient temperature while the user does not recognize, and it is possible to measure temperature, skin condition and a particular smell component. When a human body is a detection object, temperature change is small while measuring a smell without touching, for example, measuring a breath odor, and in contrast, a temperature change is large due to a body temperature while measuring a skin condition by directly touching, for example, measuring amount of moisture. Therefore, it is possible to measure multiple physical quantities by using a single sensor because switching the switch means SW based on the thermocouple which detects the temperature change. Therefore, it is possible to reduce the number of components, a circuit area and a number of wirings, and in addition, and in addition, it is possible to comprehensively check skin condition, body condition and mental condition that are not easy for a conventional single detection element. (Example)

A sensor 1 shown in FIG. 1A was produced. An outside dimension of a substrate 8 applied to the sensor 1 includes each edge of 10 (mm) and a thickness (T) of 1 (mm). The material is glass-ceramics, and an engaged comb electrode 2 is formed on a surface of the substrate 8. A size of the engaged comb electrode 2 shown in FIGS. 5A and 5B includes: 7 (mm) of width (W); 7 (mm) of length (L); 0.5 (mm) of electrode width (a); 0.5 (mm) of distance between electrodes (p); and 7 (pairs), that are, the number of pairs (n) of electrodes. A thermocouple is formed by using copper, copper nickel alloy to both the horizontal electrode 21 and the vertical electrode 23. Further, the sensitive membrane 3 is formed at a portion of the engaged comb electrode 2. The sensitive membrane 3 is formed so as to cover the electrodes which are a pair. Further, the switch SW is attached to a center portion of the horizontal electrode 22. (Second embodiment)

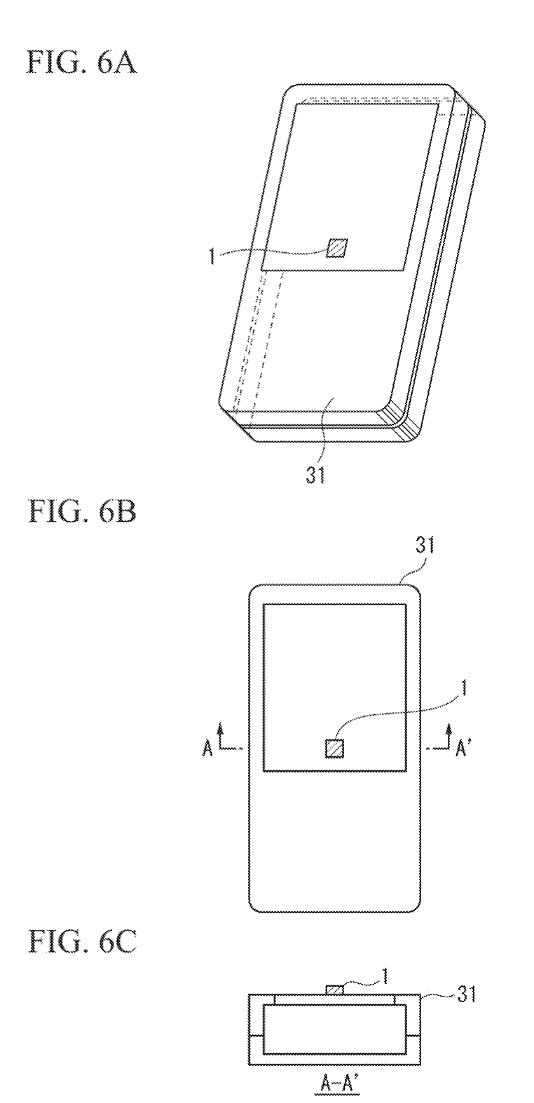

FIGS. 6A-C are a perspective drawing, a plane figure and a cross-sectional figure along with a line between A-A′ that show a constitution of an electric apparatus (mobile terminal) of the second embodiment. In these drawings, a reference numeral 1 is a sensor shown in FIG. 1A, and a reference numeral 31 is an electric apparatus. A chassis size of the electric apparatus 31 includes: 50 (mm) of width (W); 90 (mm) of length (L); and 10 (mm) of height (T). As shown in the drawings, the sensor 1 is mounted at an approximately a center portion of an outside surface on a side of a receiver of the electric apparatus 31 by adhering. In this embodiment, the substrate 8 (FIGS. 1A and 1B) is mounted on the electric apparatus 31 by adhering, and it is possible to form the engaged comb electrode 2 directly on a surface of the chassis of the electric apparatus 31.

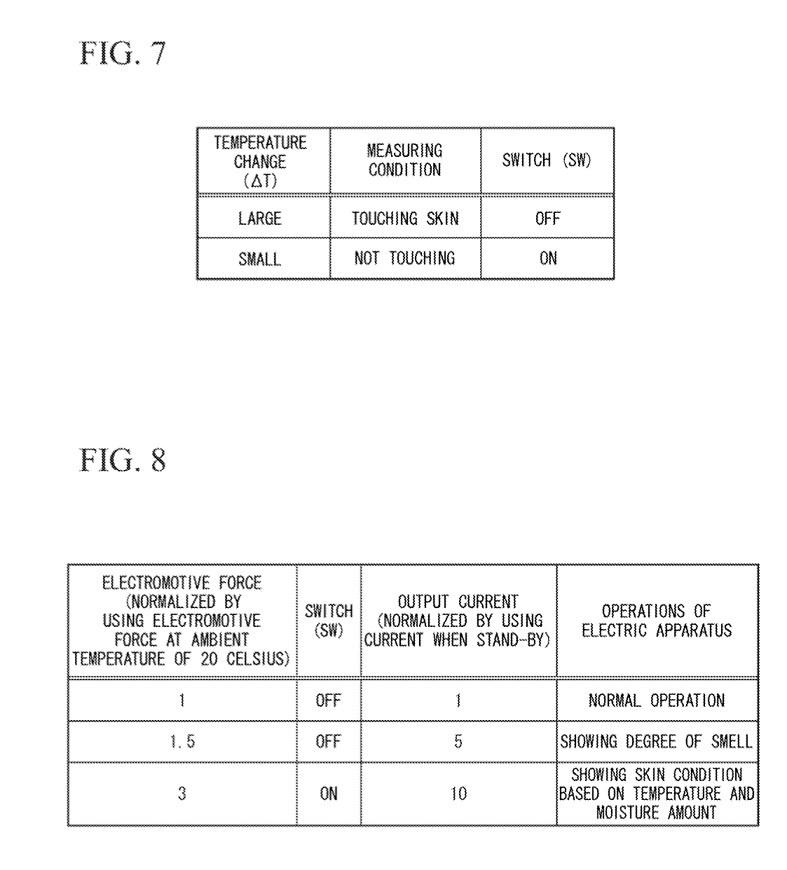

The sensor 1 measures temperature, amount of moisture and a specific smell component by measuring an electromotive force, capacitance and an electric resistance between the electrode terminals 5a, 5b and 5c, and here, a constitution is optimized so as to be preferable for measuring skin condition of a person and a breath odor. The sensor 1 measures temperature by measuring an electromotive force caused by the thermocouple by measuring a potential difference between the electrode terminals 5a-5c Here, the electrode terminal 5c is connected to the switch means SW, and a switching operation is conducted between the skin condition detection portion 16 and the smell component detection portion 17 in accordance with a comparison result between the electromotive force generated by a temperature change and a predetermined threshold. FIG. 7 shows connection state with skin that is assumed based on the voltage changes of the thermocouple, operations of the switch means (switch) SW and measured items. If the voltage change of the thermocouple is minimal, it is determined that the sensor 1 and skin are in a non-contact state. In such a state, the switch means (switch) SW is OFF, and the smell component and its concentration are measured based on the resistance change r1 of the resistance R1 shown in FIG. 2. Here, the smell component which is measured depends on the sensitive membrane 3. On the other hand, when the electromotive force of the thermocouple increases, it is determined that the sensor 1 and skin are in a contact state because there is a possibility of temperature increase due to skin temperature. In such a case, the skin conditions, for example, the amount of moisture, is measured based on the capacitance fluctuation C by setting the switching means (switch) SW ON. It is natural that the measured temperature is used as data which indicates the skin condition too. In accordance with above-described operations, it is possible to select a circuit constitution which has high sensitivity in accordance with conditions when measuring, and it is possible to measure the skin conditions and the smell component while the user does not recognize.

An operation example of this embodiment is shown in FIG. 8. When the electromotive force (normalized by using the electromotive force at an ambient temperature of 20 Celsius) is “1”, the switching means (switch) SW is OFF, and when the output current (normalized by using an current when stand-by) obtained at the electrode terminal 5b is “1”, an electric apparatus (mobile terminal) 31 conducts a normal operation. Further, when the electromotive force is “1.5”, and when the output current is “5”, the switching means (switch) SW is OFF, and electric apparatus 31 shows a degree of smell. When the electromotive force is “3”, and when the output current is “10”, the switching means (switch) SW is ON, and electric apparatus 31 shows the skin condition based on both the temperature and the amount of moisture. (third embodiment)

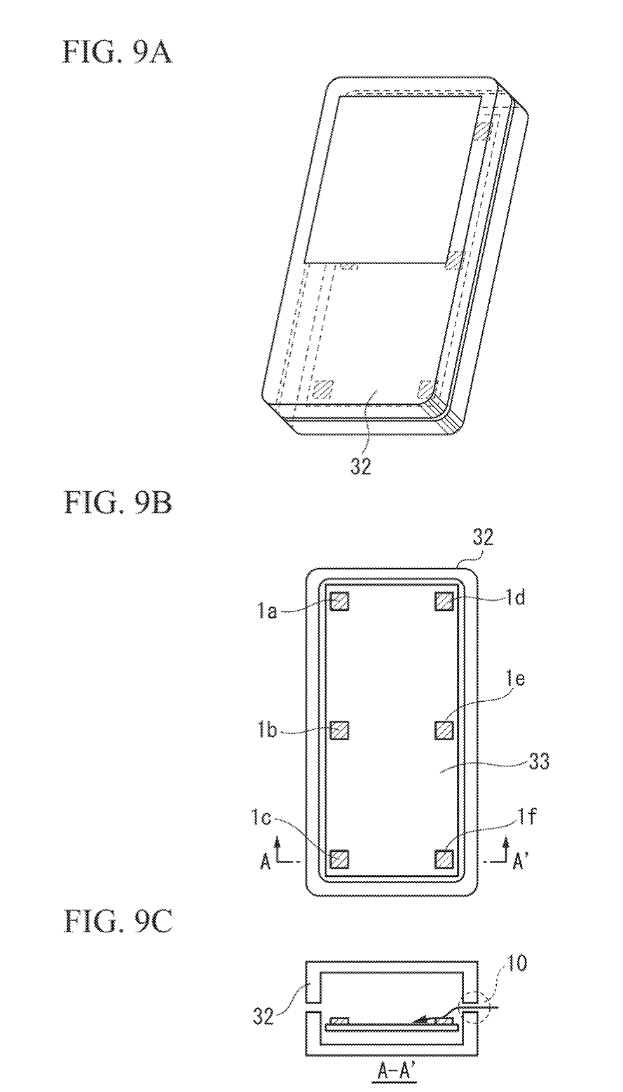

FIGS. 9A-C are a perspective drawing, a plane figure and a cross-sectional figure along with a line between A-A′ that show a constitution of an electric apparatus (mobile terminal) of the third embodiment. In these drawings, referenced numerals 1a-1f are sensors shown in FIG. 1A, and 32 is an electric apparatus. The sensors 1a-1f are mounted on a circuit substrate 33 of the electric apparatus 32. Mounting positions are, for example, an easily immersible position 13 and positions which are concerned because of heat and smoke caused by a rechargeable battery. Further, each switching means SW (see FIG. 1A) of the sensors 1a-1f is set to OFF when an initial state.

Hereinafter, operations of the third embodiment are explained. Each of sensors 1a-1f which measures temperature, amount of moisture and a specific smell component by measuring a potential difference between the electrode terminals 5a and 5c and by measuring both a capacitance and an electric resistance between the electrode terminals 5a and 5b, conducts both detection of immersion and detection of heat and smoke particularly in such a constitution.

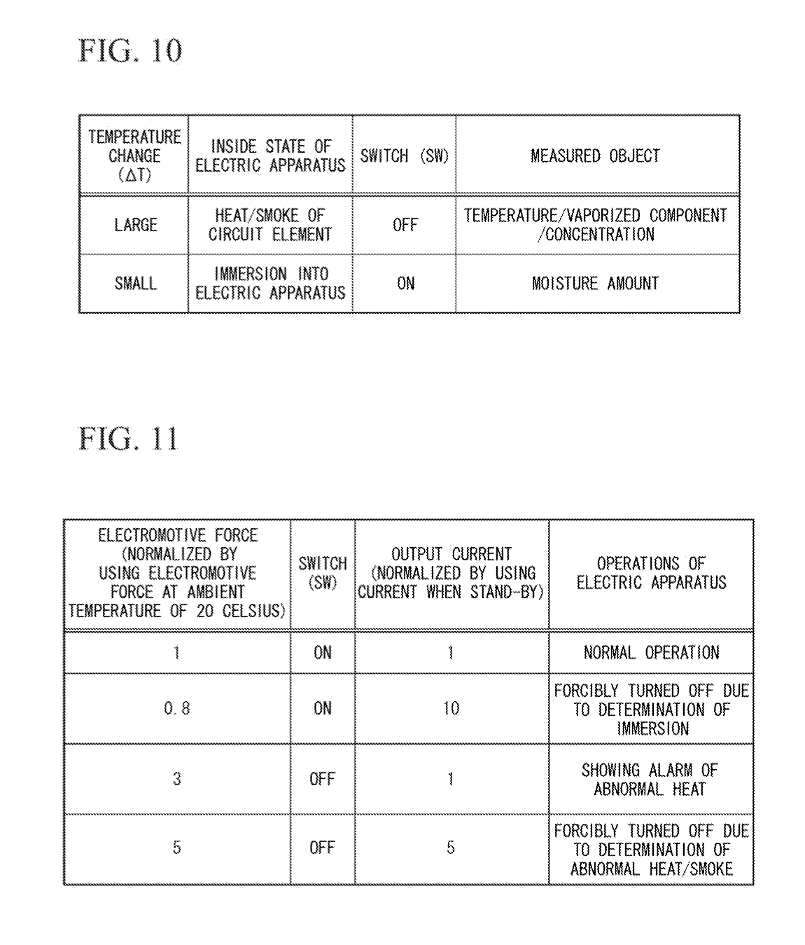

In other words, the electromotive force caused by each of thermocouples of the sensors 1a-1f is measured by measuring the potential difference between the electrode terminals 5a and 5c, and in accordance with such an operation, the temperature is measured. Here, the electrode terminal 5c is connected to the switching means SW, and therefore, the skin condition detection portion 61 and the smell component detection portion 17 are switched based on a comparison result between the electromotive force caused by the temperature changes and a predetermined threshold. It should be noted that the skin condition detection portion 16 of such a constitution detects amount of moisture adhered to the skin condition detection portion 16, that is, whether or not there is an immersion inside the electric apparatus 32. Internal conditions of the electric apparatus 32 that are predicted based on the changes of the voltage of the thermocouples, operations of the switching means SW and measuring items are shown in FIG. 10. If the voltage change of the thermocouple is minimal, it is possible to determine that there is no abnormal heat from the circuit elements. In such a case, the sensors 1a-1f function only as immerse detection sensors and conduct immerse detection inside the electric apparatus 32 by using the capacitance fluctuation c of the skin condition detection portion 16.

On the other hand, if the electromotive force of the thermocouples is increased, temperature increase around circuit components caused by an abnormal heat of the circuit elements is assumable. When detecting such a temperature increase, the switching means SW is set to OFF, the smell component is measured based on the resistance change r1 of the resistance R1 of the smell component detection portion 17. When the abnormal heat of the circuit elements is continued, a flax included in a protection layer and a solder paste of the circuit substrate vaporized, in accordance with the smell component detection portion 17 using a component which is the sensitive membrane 3 that is selective, an abnormal condition is detected based on both temperature increase and the smell component simultaneously or gradually, and an appropriate operation is conducted. In this embodiment, a means is provided which forcibly turns off the power of the electric apparatus 32 when the immersion into the electric apparatus 32 is detected if the temperature increase is lower than a predetermined threshold and if the output current is higher than a predetermined threshold. Further, when the temperature increase is higher than the predetermined threshold, an alarm of abnormal heat is shown on a screen display for the user, and in addition, a means is provided which forcibly turns off the power of the electric apparatus 32 when both the temperature increase and the smell component are simultaneously detected.

An operation example of this embodiment is shown in FIG. 11. When the electromotive force (normalized by using the electromotive force at an ambient temperature of 20 Celsius) is “1”, the switching means (switch) SW is ON, and when the output current (normalized by using an current when stand-by) obtained at the electrode terminal 5b is “1”, an electric apparatus 32 conducts a normal operation. Further, when the electromotive force is “0.8”, the switching means (switch) SW is ON. In such a condition, if an output current is “10”, the electric apparatus 32 determines an immersion is occurred and forcibly turns off the power. When the electromotive force is “3”, the switching means (switch) SW is set to OFF. In such a state, when the output current is “1”, the electric apparatus 32 displays abnormal heat. When the electromotive force is “5”, the switching means (switch) SW is set to OFF. In such a state, when the output current is “5”, the electric apparatus 32 forcibly turns off the power because of determining abnormal heat and smoke.

In accordance with the above-described third embodiment, it is possible to detect immersion and heat/smoke that cause a breakdown of the electric apparatus 32. Further, the temperature change is small when the immersion is detected, and on the other hand, the temperature change is large when the smell component of, for example, a flax which is vaporized due to abnormal heat of the circuit components is detected. Therefore, it is possible to measure multiple physical quantities with high sensitivity by using a single sensor because the switching means SW is operated based on the detection results of a thermocouple which detects temperature changes. Therefore, it is possible to reduce the number of components, a circuit area and a number of wirings, and in addition, and in addition, it is possible to comprehensively check skin condition, body condition and mental condition that are not easy for a conventional single detection element.

Further, it is possible to detect the immersion at immersible portions, for example, a gap between chassis portion 10, because a portion of or all functions of sensors are formed on the circuit substrate 33 of the electric apparatus 32 as shown in FIGS. 9A-C, and due to this, it is possible to avoid losing information because of immersion. Further, it is possible to detect excessive heat of a power device mounted on the electric apparatus 32 and/or of rechargeable battery and smoke based on a vaporized component of flax which is an indication before a fire. Further, an impedance between the engaged comb electrodes 2 is very high due to a constitution, electric power consumption (P) while in a stand-by mode during a measuring operation is calculated in accordance with a relation formula P=V̂2/Z by using a voltage (V) and an impedance (Z), and it is possible to operate with a low electric power consumption because electric consumption between the terminal electrodes 5a-5b is low. (Fourth embodiment)

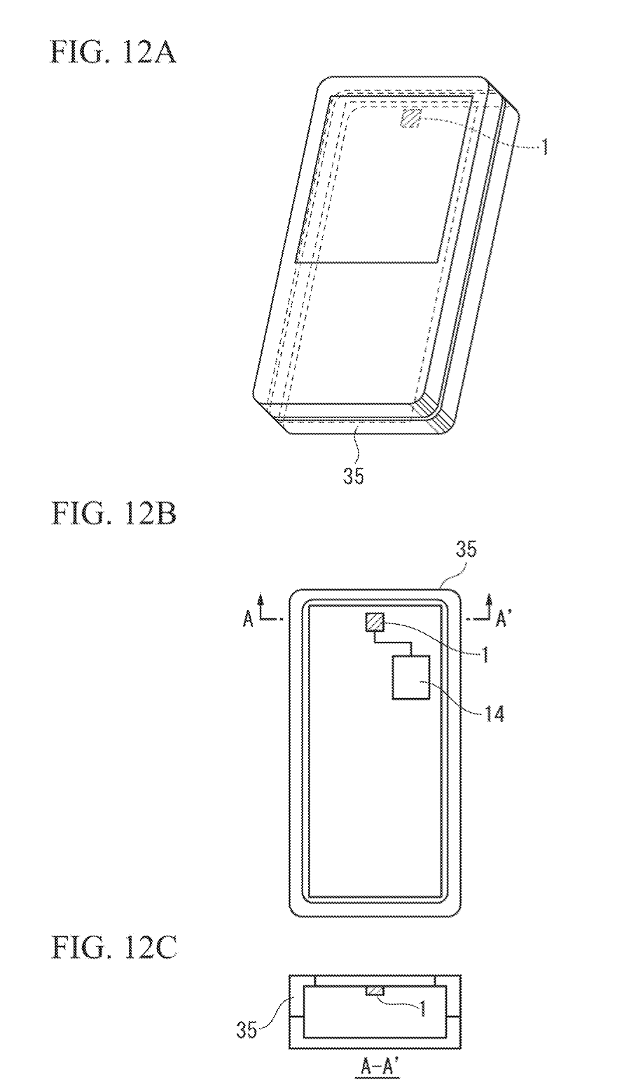

FIGS. 12A-C are a perspective drawing, a plane figure and a cross-sectional figure along with a line between A-A′ that show a constitution of an electric apparatus (mobile terminal) of the fourth embodiment. In these drawings, referenced numeral 1 is a sensor shown in FIG. 1A, 35 is an electric apparatus, and the sensor 1 is mounted on a circuit substrate of the electric apparatus 35. Mounting positions are arbitrary arranged if the sensor 1 can measure the temperature changes caused by a temperature of a person. Further, 14 is a transmitter which is mounted on the circuit substrate of the electric apparatus 35 and is connected to the switching means of the sensor 1. Further, the switching means SW of the sensor 1 is set to OFF when an initial state and is set to ON when the temperature rises.

Hereinafter, operations of this embodiment are explained.

The sensor 1 which measures amount of moisture, electric resistance, a specific smell component and temperature by measuring both a capacitance and an electric resistance between the electrode terminals 5a and 5b and by measuring a potential difference between the electrode terminals 5a and 5c, is optimized to detect a human body when touching/non-touching.

In this embodiment, the engaged comb electrodes 2 are used as a pair of transmission/reception antennas. Temperature changes caused by an approaching person are measured by the thermocouple, and when the voltage change caused by temperature increase is larger than a predetermined threshold, it is assumed that there is a human body which is a measuring object close to the sensor 1. When the voltage change caused by the temperature increase is larger than the threshold, and when the switching means SW is set to ON, the engaged comb electrodes 2 input an alternatively repeated bit signal from the transmitter 14, and the engaged comb electrodes 2 radiate electromagnetic waves. Due to this, the engaged comb electrodes 2 radiate the electromagnetic waves having frequencies which have high sensitivity for discriminating a human body, it is possible to use a pair of measured quantities including both temperature and electric impedance of a human body, and it is possible to accurately detect a human body. Further, the sensor 1 does not consume the electric power while stand-by because thermocouple applied to the sensor 1 is a passive component, and it is possible to achieve low electric consumption of the implemented electric apparatus because the electric power is consumed for radiating the electromagnetic waves for discriminating a human body only when it is measured that a human body is approaching.

### INDUSTRIAL APPLICABILITY

The above-described biological information detection sensor, an electric apparatus using the biological information detection sensor and a biological information detection method are applied to, for example, a health control, mental condition, and measuring of smells, for example, alcohol and aging odors

### DESCRIPTION OF THE REFERENCE SYMBOLS

1 and 1a-1f . . . biological information detection sensors
2 . . . engaged comb electrode
3 . . . sensitive membrane
5a, 5b and 5c . . . electrode terminals
6 . . . connection portion
7 . . . comparator circuit
8 . . . substrate
14 . . . transmitter
15 . . . temperature detection portion
16 . . . skin condition detection portion
17 . . . smell component detection portion
21 and 22 . . . horizontal electrodes
21a, 21b, 21c, 22a, 22b, 22c, 22d and 23 . . . vertical electrodes
SW . . . switching means
R1 and R2 . . . equivalent resistances
C . . . equivalent capacitance
VDC . . . electro motive force
31, 32 and 35 . . . electric apparatuses (mobile terminals)

1. A biological information detection sensor comprising:
a smell detection means which senses a specific smell;
a moisture detection means which senses moisture;
a temperature detection means which detects an ambient temperature; and
a switching means which switches between the smell detection means and the moisture detection means based on detection results of the temperature detection means.

2. The biological information detection sensor according to claim 1, wherein
the smell detection means, the moisture detection means, the temperature detection means and the switching means are formed on one substrate.

3. The biological information detection sensor according to claim 2, wherein
the substrate is formed by using a glass-ceramics.

4. The biological information detection sensor according to claim 1, wherein
the smell detection means a selective sensitive membrane which changes an electric resistance in accordance with absorbed objects.

5. The biological information detection sensor according to claim 1, wherein the moisture detection means is engaged comb electrodes comprising a first comb electrode and a second comb electrode that are arranged to be alternatively engaged while having minimal gaps.

6. The biological information detection sensor according to claim 1, wherein the temperature detection means is a thermocouple.

7. The biological information detection sensor according to claim 6, wherein the thermocouple comprises a metal which is different form the engaged comb electrode and which is connected to a means of the comb electrode.

8. The biological information detection sensor according to claim 8, wherein
the engaged comb electrode is formed by using a copper, and
the metal is a copper nickel alloy.

9. The biological information detection sensor according to claim 1, wherein the switching means is a comparator.

10. A biological information detection sensor comprising:
a skin condition detection portion measuring at least one of a capacitance and an electric resistance change between engaged comb electrodes;
a smell component detection portion measuring an electric resistance in accordance with objects absorbed on a selective sensitive membrane which is formed on a portion of the engaged comb electrodes;
a temperature detection portion measuring an ambient temperature based on a electromotive force generated by a thermocouple which is formed by using at least two kinds of metallic materials and which is formed on a portion of the engaged comb electrodes; and
a switching portion which compares the electromotive force generated by the thermocouple due to changes of the ambient temperature to a predetermined threshold, detects a contact state with an object having a heat source such as a biological body based on a comparison result and conducts switching operation of ON and OFF of the skin condition detection portion and the smell component detection portion based on the contact state.

11. The biological information detection sensor according to claim 10, wherein the skin condition detection portion measures an amount of moisture.

12. An electric apparatus comprising the biological information detection sensor according to claim 1, wherein
the electric apparatus conducts a measuring operation of a skin condition and a breath odor of a person based on detection results of the biological information detection sensor.

13. An electric apparatus comprising the biological information detection sensor according to claim 1, wherein
the biological information detection sensor is mounted at immersible points such as a gap between chassis, and
the electric apparatus detects immersion into the electric apparatus based on detection results of the biological information detection sensor.

14. An electric apparatus comprising the biological information detection sensor according to claim 1 which is mounted inside the electric apparatus, wherein
the electric apparatus simultaneously or gradually conducts both detection of abnormal heat from circuit components mounted on a circuit board and/or a rechargeable battery and detection of smoke which is an indication before a fire based on detection results from the biological information detection sensor.

15. An electric apparatus comprising the biological information detection sensor according claim 1, wherein the electric apparatus detects an approaching human body based on temperature detection results of the biological information detection sensor and detects the human body by measuring an electric impedance of the human body based on moisture detection results.

16. A biological information detection method comprising:
a detection operation of detecting a specific smell;
a moisture detection operation of detecting moisture;
a temperature detection operation of detecting an ambient temperature; and
a switching operation of switching the smell detection operation and the moisture detection operation based on detection results of the temperature detection operation.
